(19) United States
(12) Patent Application Publication
(10) Pub. No.: US 2026/0183459 A1
(43) Pub. Date: Jul. 2, 2026

(54) SYSTEM AND METHOD FOR CONTROLLING BLOOD OXYGENATION

(71) Applicant: Spectrum Medical Ltd., Gloucester, Gloucestershire (GB)

(72) Inventor: Stephen Turner, Gloucester, Gloucestershire (GB)

(21) Appl. No.: 19/130,796

(22) PCT Filed: Nov. 9, 2023
(86) PCT No.: PCT/GB2023/052933

§ 371 Date: May 16, 2025

(30) Foreign Application Priority Data
Nov. 18, 2022 (GB) 2217324.9

(51) Int. Cl. A61M 1/16; A61M 60/113; A61M 60/38

(52) U.S. Cl. CPC A61M 1/1603; A61M 1/1698; A61M 60/113; A61M 60/38; A61M 2205/3334; A61M 2230/205

(57) ABSTRACT

A system 100 for controlling blood oxygenation in a patient. The system comprises an oxygenator 200 configured to receive blood from the patient and to expose the blood to an amount 124 122 of oxygen as the blood passes through the oxygenator 200 to produce oxygenated blood. The system 100 further comprises a sensor 110 positioned downstream of the oxygenator 200 and configured to 120 measure a saturation of oxygen present in the oxygenated blood. The system 100 further comprises a controller 150. The controller 150 is configured to receive the measured saturation of oxygen from the sensor 110, calculate a difference between a target saturation of oxygen and the measured saturation of oxygen, and adjust the amount of oxygen to which the blood is exposed in the oxygenator 200 to reduce the difference.

The present disclosure relates to a system and a method for controlling blood oxygenation in a patient. In particular, the disclosure relates to a system and a method for more accurate and safe control of the saturation of oxygen in the patient's blood.

Cardiac perfusion involves extracorporeal oxygenation of a patient's blood, for example, when a patient is unable to oxygenate their own blood by breathing. Extracorporeal oxygenation involves an oxygenator that acts in place of a patient's own lungs, such as during heart and/or lung surgery.

An example of a physiological parameter relevant to blood oxygenation is the saturation of oxygen in the blood, which refers to the percentage of oxygenated haemoglobin in the patient's blood relative to the total amount of haemoglobin in the patient's blood. During extracorporeal perfusion, an oxygenator exposes the blood to an amount of oxygen as the blood passes through the oxygenator. If this amount of oxygen remains constant, then variations in the metabolic rate of the patient may lead to variations in the saturation of oxygen in the blood. For example, a patient may experience a low metabolic rate. The patient's metabolic rate may be deliberately reduced (e.g. by reducing the patient's temperature), before placing the patient into a state of circulatory arrest, for example, in order to perform surgery on the patient's heart. The patient's body will consume less oxygen but the oxygenator will continue to supply a constant amount of oxygen to the blood. This leads to an increase in the saturation of oxygen and leads to an increased risk of over-oxygenation (i.e. hyperoxia).

Conventionally, when controlling oxygenation in the blood, clinicians may directly measure a variety of physiological parameters, such as the partial pressure of oxygen in the patient's arterial blood (abbreviated as ‘PaO2’). The clinician may then treat some or all of the physiological parameters (e.g. partial pressure of oxygen) as a ‘proxy’ for the saturation of oxygen in the patient's blood. That is, clinicians will typically rely on changes in and/or the absolute value of partial pressure of oxygen as an indicator of changes in and/or the absolute value of the saturation of oxygen in the patient's blood. Control of the patient's blood oxygen level is thus conventionally achieved by controlling and monitoring the partial pressure of oxygen, acting as a proxy for the saturation of oxygen.

However, there is a considerable issue with controlling oxygenation based on partial pressure. Specifically, the saturation of oxygen in the patient's blood will reach 100% saturation at a particular partial pressure of oxygen (e.g. 100-125 mmHg) and any further increase in the partial pressure will no longer impact the saturation of the blood. Therefore, monitoring the partial pressure of oxygen leads to a conventional practice in which a clinician will deliberately maintain a partial pressure of oxygen that is ‘too high’ (e.g. 200-300 mmHg) to ensure that 100% oxygen saturation is achieved. (For comparison, a healthy partial pressure of oxygen is considered to be in the range of 75-100 mmHg.)

While this conventional approach may reduce the risks associated with under-oxygenation (i.e. hypoxia), it exposes the patient to the lesser-known risks associated with hyperoxia, which may, in particular, arise if a patient's metabolic rate drops during a surgical procedure (e.g. during circulatory arrest).

The conventional approach can therefore cause considerable physiological harm for such patients. Exposing such a patient's blood to high levels of oxygen can lead to hyperoxia and an increased risk of oxidative stress, which in turn can lead to increased risks of disorders in the patient's cardiovascular or neurological systems (e.g. cardiac arrest and seizures).

The conventional approach can also cause physiological harm to patients who are suffering of low saturation of oxygen in the blood. For example, a particular demographic of patient that can suffer from these detrimental effects are cyanotic patients. A cyanotic patient is a patient who is suffering from low saturation of oxygen in the blood, which typically leads to a blue discolouration of the skin. The condition more commonly occurs among babies, particularly those with congenital heart abnormalities.

Therefore, there is a need to provide a system and method for controlling blood oxygenation with improved accuracy and safety.

It will be noted that, for accuracy and in line with convention in the medical field, values of pressure given herein are provided in units of mmHg (‘millimetre of mercury’). However, these values may be simply converted to atm (‘atmospheres’) in noting that 1 atm is equal to 760 mmHg, or to Pa (‘Pascal’) in noting that 1 mmHg is approximately equal to 133.3 Pa.

In accordance with a first aspect, there is provided a system for controlling blood oxygenation in a patient, as defined in claim 1.

The system comprises an oxygenator configured to receive blood from the patient and to expose the blood to an amount of oxygen as the blood passes through the oxygenator to produce oxygenated blood. The system further comprises a sensor positioned downstream of the oxygenator and configured to measure a saturation of oxygen present in the oxygenated blood. The system further comprises a controller. The controller is configured to receive the measured saturation of oxygen from the sensor, calculate a difference between a target saturation of oxygen and the measured saturation of oxygen, and adjust the amount of oxygen to which the blood is exposed in the oxygenator to reduce the difference.

Advantageously, the sensor measures the saturation of oxygen in the blood following oxygenation by the oxygenator. That is, the sensor being ‘positioned downstream of the oxygenator’ means that the blood flows past the sensor after having passed through the oxygenator. In other words, the sensor is positioned downstream with respect to the direction of blood flow. In this regard, the saturation of oxygen measured by the sensor may be referred to as the ‘arterial oxygen saturation’. This is in contrast to ‘venous oxygen saturation’, which would be measured upstream of the oxygenator (for example, immediately after the blood exits the patient or between a venous reservoir and the oxygenator).

Venous oxygen saturation is heavily influenced by physiological factors of the patient. For example, venous oxygen saturation is a function of the patient's metabolic rate, blood flow, haemoglobin levels, and other physiological parameters. Measuring the venous oxygen saturation therefore inherently incorporates uncertainty and a lack of accuracy into any subsequent control of the patient's blood oxygen level based thereon. A clinician would need an understanding of anaesthetic factors, the degree of paralysis of a patient, the patient's temperature, the state of a patient's capillary beds, autonomic responses (e.g. immune or inflammatory reactions), and a variety of other factors in order to control oxygen saturation based on venous oxygen saturation measurements. Therefore, using a sensor to measure the saturation of oxygen following oxygenation provides increased accuracy and safety of the system for controlling blood oxygenation and saturation.

As used herein, the term ‘saturation of oxygen’ refers to the percentage of oxygenated haemoglobin in the patient's blood relative to the total amount of haemoglobin in the patient's blood. The terms ‘saturation of oxygen’ and ‘oxygen saturation’ are used interchangeably herein. The sensor being configured to ‘measure the saturation of oxygen’ means that the sensor produces a value that may be interpreted by the controller as directly representing the saturation of oxygen. For example, the sensor may output a digital value equal to the saturation of oxygen measured by the sensor, or the sensor may output an analogue value (e.g. a voltage) that is related to (e.g. proportional to) the saturation of oxygen measured by the sensor. It is to be appreciated that the measurement of the saturation of oxygen (e.g. by a sensor) is different from the calculation of the saturation of oxygen, which would involve the measurement of auxiliary physiological parameters (e.g. partial pressure of oxygen) and deriving the saturation of oxygen by applying mathematical operations to these auxiliary physiological parameters. The term ‘adjust’ refers to increasing or decreasing a parameter.

Advantageously, the controller adjusts the amount of oxygen based on a measured value of the saturation of oxygen to reduce the difference between the target saturation and the measured saturation. This control by the controller may be referred to as ‘closed-loop’ control. The closed-loop control is performed on the measured saturation of oxygen, rather than on a proxy for this value (such as partial pressure of oxygen). In this regard, the present invention can be considered to relate to a ‘direct’ closed-loop control of the saturation of oxygen, compared to the conventional ‘indirect’ closed-loop control.

The controller may be configured to perform some or all of these operations continuously. For example, the controller may be configured to continuously receive the measured saturation, continuously calculate the difference, and/or continuously adjust the amount of oxygen. By ‘continuously’, it is meant that the operation may be performed in an ongoing manner with no interruptions. Alternatively or additionally, the controller may be configured to perform some or all of these operations repeatedly. For example, the controller may be configured to repeatedly receive the measured saturation, repeatedly calculate the difference, and/or repeatedly adjust the amount of oxygen. By ‘repeatedly’, it is meant that the operation may be performed in recurring discrete intervals (e.g. once a millisecond, once a second, once a minute).

By providing closed-loop control of a measured oxygen saturation, several advantages are realised. For example, the system is able to more accurately maintain the saturation of oxygen and is also able to respond to changes in a patient's metabolic rate by adjusting the amount of oxygen to which the blood is exposed in response to changes in the saturation of oxygen. This is particularly beneficial in the treatment of patient who experience a drop in metabolic rate (e.g. patients in circulatory arrest). In addition, the system is able to accurately maintain the saturation of oxygen at a lower level, because there is no need to overcompensate to maintain safe saturation levels. This allows the system to be particularly beneficial in the treatment of patients with low blood oxygen levels (e.g. cyanotic patients). That is, the system may be used to first match a target saturation of oxygen (e.g. the patient's current lower saturation level) using the closed-loop control, before slowly adjusting (e.g. increasing) the saturation of oxygen in order to establish a healthy saturation of oxygen. For example, the target saturation of oxygen may be taken as a typical saturation of oxygen in a healthy patient, such as between 95-100%, between 98-99%, or approximately 98.5% oxygen saturation. Advantageously, an oxygen saturation of 98.5% is approximately the saturation of oxygen in the blood of a healthy patient. Furthermore, choosing a saturation of oxygen that is below 100% allows ‘headroom’ to increase the saturation further if required.

The controller may be configured to receive the target saturation of oxygen. More specifically, the target saturation of oxygen may be received by the controller as an input. For example, the controller may be configured to receive the target saturation of oxygen as an input from a user interface. Alternatively or additionally, the controller may be pre-programmed with a target saturation of oxygen. Alternatively or additionally, the controller may be configured to retrieve the target saturation of oxygen from a lookup table stored locally or on a remote server.

It will be appreciated that, in some embodiments, the sensor may be configured to measure a physiological parameter other than the saturation of oxygen. That is, while the measurement of the saturation of oxygen is preferable for the reasons described, more generally, the sensor may be configured to measure a physiological parameter indicative of the oxygenation of oxygenated blood. In such embodiments, the controller may also be configured to perform any or all of the operations described herein (particularly such operations related to the control of the oxygenation gas present in the oxygenator). The operations may be performed with respect to controlling the measured physiological parameter, rather than specifically the saturation of oxygen. For example, the physiological parameter may comprise the partial pressure of oxygen. In some embodiments, the sensor is positioned on a blood line, the blood line being connected to the oxygenator and configured to carry the oxygenated blood from the oxygenator to the patient.

In other words, the sensor may be positioned between the oxygenator and the patient. That is, the sensor may be positioned upstream of the patient. The blood line may directly connect the oxygenator to the patient. In such an example, the sensor may be integral with the oxygenator (e.g. integral with a blood outlet of the oxygenator) or the sensor may be separate from the oxygenator and connectable to the blood line. Alternatively, additional components of the system (e.g. sensors, fluid delivery devices, valves and/or actuators) may be located between the oxygenator and the sensor and/or between the sensor and the patient.

Advantageously, by positioning the sensor on a blood line configured to carry the oxygenated blood from the oxygenator to the patient, the sensor is configured to measure the ‘arterial saturation of oxygen’ (abbreviated as ‘SaO2’). SaO2 is defined as the saturation of oxygen in the arterial blood of a patient as measured directly from the blood. In the field of cardiac perfusion, SaO2 is considered to be the ‘true’ value of the saturation of oxygen in the arterial blood of a patient. This is in contrast to the so-called ‘peripheral’ saturation of oxygen in the arterial blood, abbreviated as ‘SpO2’. SpO2 is considered to be an estimate of SaO2 and is measured using pulse oximetry.

Pulse oximetry involves placing a device, called a pulse oximeter, onto a peripheral part of the patient's body (e.g. ear, finger, toe, hand, foot) and measuring the saturation of oxygen through the patient's skin. The pulse oximeter may comprise a light-emitting component and a light sensor, wherein the light-emitting component shines light of varying wavelengths through the patient's skin into a blood vessel and the light sensor detects the reflected light to determine the level of oxygen present in the patient's blood. In this regard, pulse oximetry is not performed on a blood line configured to carry the oxygenated blood from the oxygenator to the patient, since it is performed directly on the patient. Thus, the saturation measured using pulse oximetry is considered to be less accurate because the patient's body may have already absorbed an unknown quantity of oxygen before the blood reaches the point on the body at which the saturation is measured using pulse oximetry. Furthermore, measurement of saturation through the patient's skin, as is the case in pulse oximetry, introduces further inaccuracy due to, for example, variation in the light absorption of the patient's skin. Therefore, SpO2 is considered to be merely an estimate of SaO2 and thus positioning the sensor on a blood line configured to carry the oxygenated blood from the oxygenator to the patient has the effect of allowing a more accurate value for the saturation of oxygen to be obtained. This, in turn, allows for more accurate control of blood oxygenation in the patient.

In some embodiments, the sensor is configured to measure the saturation of oxygen present in the oxygenated blood via spectrophotometry performed on the blood line.

Advantageously, spectrophotometry provides an accurate means of measuring the saturation of oxygen in the blood. Furthermore, spectrophotometry is a non-invasive method that may be performed during surgery without contacting the patient's blood (e.g. to measure pH or temperature). Performing spectrophotometry on the blood line further improves the accuracy of the measurement because, as discussed above, the measurement is performed directly on the blood itself and thus provides a measurement of SaO2 (rather than SpO2 measured through the patient's skin via pulse oximetry). Spectrophotometry involves a white light source being shone towards the blood in the blood line. The white light is at least partially reflected by the blood and the reflected light is detected by a receiver. The receiver will only detect wavelengths that are reflected by the blood, and will not detect wavelengths that are absorbed by the blood. Some wavelengths in the white light are absorbed more strongly by oxygenated haemoglobin and others are absorbed more strongly by de-oxygenated haemoglobin. By measuring the absorption of each wavelength and comparing the relative absorptions, the proportion of oxygenated haemoglobin to de-oxygenated haemoglobin can be determined, which can, in turn, be used to determine the saturation of oxygen in the blood.

To assist in the spectrophotometry, the blood line may be transparent. For example, the blood line may be transparent to each of the wavelengths of light used in the spectrophotometry.

Alternatively, the sensor may be configured to measure the saturation of oxygen present in the oxygenated blood via an invasive method performed on the blood line. In the case of either invasive or non-invasive methods, the sensor may comprise a cuvette.

In some embodiments, the controller is configured to adjust the amount of oxygen by adjusting the concentration of oxygen in an oxygenation gas received by the oxygenator.

The oxygenator may be configured to expose the blood to the oxygenation gas to produce oxygenated blood. The oxygenation gas may comprise a mixture of oxygen with nitrogen and/or carbon dioxide. For example, the oxygenation gas may comprise a mixture of nitrogen and oxygen (but no carbon dioxide). Adjusting the concentration of oxygen in the oxygenation gas may comprise adjusting the relative proportions of nitrogen, carbon dioxide, and/or oxygen in the oxygenation gas. The ‘concentration of oxygen in the oxygenation gas’ may equivalently be referred to as the ‘fraction of inspired oxygen’ (abbreviated as ‘FiO2’). The controller may be configured to communicate with one or more valves or actuators that control the gas supply to the oxygenator in order to adjust the concentration of oxygen in the oxygenation gas.

The ability to adjust the concentration of oxygen in the oxygenation gas received by the oxygenator can improve the system's effectiveness in preventing hyperoxia. By way of explanation, a patient's metabolic activity can greatly slow down during cardiac perfusion. When metabolic activity is reduced, supplying pure oxygen to the oxygenator may cause the patient's oxygen saturation to become elevated, even when the flow rate of pure oxygen is low. By adjusting the concentration of oxygen in the oxygenation gas, the system can maintain the target saturation of oxygen even when the patient's metabolic activity is reduced.

The system may further comprise a gas blender configured to receive one or more supply gases and to blend the supply gases to produce one or more oxygenation gases for supplying to the oxygenator. For example, the gas blender may be configured to receive an oxygen gas supply, a nitrogen gas supply, and/or a carbon dioxide gas supply. The oxygen gas supply may comprise a pure oxygen gas supply (i.e. the oxygen gas supply consists of 100% oxygen). The nitrogen gas supply may comprise a pure nitrogen gas supply (i.e. the nitrogen gas supply consists of 100% nitrogen). Alternatively, the nitrogen gas supply may comprise a gas mixture comprising nitrogen (e.g. the nitrogen gas supply may be air, or may comprise air). The carbon dioxide gas supply may comprise a pure carbon dioxide gas supply (i.e. the carbon dioxide gas supply consists of 100% carbon dioxide). Alternatively, the carbon dioxide gas supply may comprise a gas mixture comprising carbon dioxide (e.g. the carbon dioxide gas supply may be air, or may comprise air). The gas blender may be configured to blend the oxygen gas supply with the nitrogen gas supply and/or the carbon dioxide gas supply to produce one or more oxygenation gases. It will be noted that the blending of supply gases may produce oxygenation gases that consist solely of one of the supply gases (e.g. the gas blender may supply an oxygenation gas that consists of 100% oxygen). The controller may be configured to adjust the concentration of oxygen in, and/or the flow rate of, the oxygenation gas by controlling the gas blender to do so.

It will be appreciated that the oxygenation gases may comprise additional gas components. For example, the oxygenation gas may additionally comprise one or more anaesthetic gases (e.g. isoflurane, sevoflurane, desflurane, and/or nitrous oxide). The oxygenation gases may additionally or alternatively comprise other gases common in the art (e.g. helium and/or argon).

In some embodiments, the controller is configured to adjust the amount of oxygen by adjusting the flow rate of an oxygenation gas received by the oxygenator.

It will be appreciated that adjusting the flow rate of the oxygenation gas has the effect of adjusting a volume of oxygenation gas present in the oxygenator at a given time. Therefore, adjusting the flow rate of the oxygenation gas adjusts a volume of oxygenation gas to which the blood is exposed in the oxygenator. For an oxygenation gas comprising oxygen, this adjusts the amount of oxygen to which the blood is exposed in the oxygenator. For example, increasing the flow rate of the oxygenation gas increases the volume of oxygenation gas present in the oxygenator, thereby increasing the amount of oxygen to which the blood is exposed in the oxygenator.

The controller may be configured to adjust both the concentration of oxygen in the oxygenation gas and the flow rate of the oxygenation gas. For example, the controller may be configured to adjust the concentration of oxygen in the oxygenation gas and the flow rate of the oxygenation gas concurrently.

In some embodiments, the oxygenation gas consists of pure oxygen.

Advantageously, an oxygenation gas consisting solely of oxygen does not contain any nitrogen. Nitrogen is a primary component of gaseous microemboli (GME), or gas bubbles, in the blood that passes through the oxygenator. In essence, nitrogen fails to dissolve in the blood and thus leads to the formation of GME. These GME can grow in size by accumulating a layer of blood-borne materials (e.g. proteins in the blood) on their outer surface. The GME thus acts as an obstruction in the blood and can lead to serious complications, such as tissue or organ damage. The concentration of nitrogen in the oxygenation gas is too high when the partial pressure of nitrogen in the oxygenation gas is similar to, or substantially equal to, the partial pressure of nitrogen in the blood. This leads to reduced (or substantially no) removal of nitrogen from the blood when the blood passes through the oxygenator due to a lack of diffusion gradient between the nitrogen in the blood and the nitrogen in the oxygenation gas. Consequently, due to the high nitrogen content of the blood, there is also a lack of diffusion gradient between the nitrogen in the GME and the nitrogen in the blood itself. Therefore, there is little or no diffusion of nitrogen from the GME to the blood or from the blood to the oxygenation gas, and thus, there is an increased risk of GME continuing through the bloodstream, unchanged by the oxygenator. By providing an oxygenation gas of pure oxygen, a larger diffusion gradient is created between the blood and the oxygenation gas, which in turn leads to a larger diffusion gradient between the GME and the surrounding blood. Therefore, more nitrogen transfers from the blood to the oxygenation gas, and more nitrogen transfers from the GME to the blood. The removal of nitrogen is thus maximised and thus the risk of GME is considerably or completely reduced.

In some embodiments, the oxygenator comprises a gas-blood interface which is configured to be supplied with an oxygenation gas to expose the blood to the amount of oxygen. The controller may, optionally, be configured to adjust the amount of oxygen by adjusting the proportion of the gas-blood interface that is supplied with the oxygenation gas.

The term ‘gas-blood interface’ refers to a component of the oxygenator that permits gas exchange between a gas supplied to the gas-blood interface and blood supplied to the gas-blood interface. By adjusting the proportion of the gas-blood interface that is supplied with the oxygenation gas, the system is able to control exposure of the blood to the oxygenation gas. That is, as the blood passes through the oxygenator, the blood passes through a proportion of the gas-blood interface that is supplied with the oxygenation gas and a proportion of the gas-blood interface that is not supplied with the oxygenation gas. The blood is thus only able to exchange gas (and thus be oxygenated) in a certain proportion of the gas-blood interface.

Advantageously, this provides a mechanism for adjusting the amount of oxygen to which the blood is exposed that is independent of the composition of the oxygenation gas. This means that the oxygenator can be used with standard gas supply lines, for example, that may already be installed in an operating theatre. In such examples, the oxygenation gas may comprise a mixture of oxygen and nitrogen, or may consist of pure oxygen. In the former case, the amount of oxygen to which the blood is exposed may be adjusted by adjusting the proportion of the gas-blood interface that is supplied with the oxygenation gas and/or by adjusting the concentration of oxygen in the oxygenation gas. In the latter case, the amount of oxygen to which the blood is exposed may be adjusted by adjusting the proportion of the gas-blood interface that is supplied with the oxygenation gas, while maintaining a supply of pure oxygen, and thus reducing the risk of GME, as described above.

The gas-blood interface may comprise one or more hollow fibre groups. Each hollow fibre group may comprise a plurality of hollow fibres. Each hollow fibre group may take the form of a hollow fibre bundle, a hollow fibre mat, a hollow fibre spiral, or other hollow fibre configurations known in the art.

Each hollow fibre within the hollow fibre group may comprise an opening for receiving the oxygenation gas. Each hollow fibre may comprise a gas-permeable wall that allows the exchange of gas between the blood and the oxygenation gas. The hollow fibre groups may be arranged to traverse (e.g. perpendicularly) the flow of blood as the blood passes through the oxygenator in order to expose the blood to the oxygenation gas present in the hollow fibre groups.

In some embodiments, the gas-blood interface comprises a plurality of interface regions that are each configured to be independently supplied by a respective oxygenation gas. The controller may be configured to adjust the proportion of the gas-blood interface that is supplied with oxygenation gas by altering the number of interface regions that are supplied with oxygenation gas.

The plurality of interface regions may be arranged successively with respect to the direction of blood flow through the oxygenator. That is, as the blood passes through the oxygenator, the blood passes through each interface region, one after the other.

By ‘independently supplied’ it is meant that the composition (e.g. gas concentrations) and/or flow conditions (e.g. flow rate) of each gas supply can be controlled independently for each interface region. The controller may be configured to alter the number of interface regions that are supplied with the oxygenation gas by switching on or off one or more of the independent gas supplies. For example, the controller may be configured to open or close one or more valves that allow or prevent the flow of gas from an independent gas supply into a respective interface region. In such an example, the gas-blood interface may comprise a plurality of hollow fibre groups, each hollow fibre group corresponding to a respective one of the interface regions. Alternatively, the gas-blood interface may comprise a single hollow fibre group, each interface region corresponding to a portion of the single hollow fibre group.

Alternatively or additionally, the controller may be configured to independently adjust the composition and/or the flow rate of the oxygenation gas supplied to each of the interface regions. That is, more than one of the interface regions may be supplied with oxygenation gas, but the composition and/or the flow rate of this oxygenation gas may be varied for each of the interface regions independently of the composition and/or the flow rate in the other interface regions.

The ability to independently adjust the composition and/or the flow rate of the oxygenation gas supplied to each of the interface regions can further improve the system's effectiveness in preventing hyperoxia in patients whose metabolic activity is reduced. For example, the system can maintain the target saturation of oxygen in such patients by reducing (or even stopping) the flow rate of the oxygenation gas supplied to one interface region whilst also reducing the concentration of oxygen in the oxygenation gas supplied to another interface region. The system can allow numerous permutations of the composition and/or flow rate of the oxygenation gas supplied to each of the interface regions, thus enabling effective control of oxygen saturation in a wide variety of clinical situations.

In some embodiments, the oxygenator comprises a gas inlet zone for receiving oxygenation gas into the gas-blood interface from each independent gas supply. The gas inlet zone may comprise one or more partitions dividing the gas inlet zone into a plurality of gas inlet regions. Each gas inlet region may be configured to receive oxygenation gas from a different one of the respective oxygenation gases. Each gas inlet region may be configured to provide the oxygenation gas to the respective interface region.

In such an example, the gas-blood interface may comprise a plurality of hollow fibre groups, each hollow fibre group corresponding to one of the interface regions, and thus to one of the gas inlet regions. Alternatively, the gas-blood interface may comprise a single hollow fibre group, and each interface region (and thus each gas inlet zone) may correspond to a portion of the single hollow fibre group.

The one or more partitions may separate the gas-blood interface. For example, a partition may extend from the gas inlet zone and into the gas-blood interface, and may extend through the gas-blood interface. Where the gas-blood interface comprises a plurality of hollow fibre groups, the hollow fibre groups may be separated from one another by a gap in which a partition is located.

At least one of the one or more partitions may be movable. For example, the one or more partitions may be movable to adjust the relative sizes of each of the gas inlet regions. The controller may be configured to adjust the positions of the one or more partitions (e.g. by activating a motor coupled to the one or more partitions). For example, the controller may be configured to receive an input representative of a desired saturation of oxygen and/or a desired position of the one or more partitions. The controller may further be configured to adjust the positions of the one or more partitions responsive to the input. The controller may further be configured to convert a desired saturation of oxygen into a position of the one or more partitions. For example, by adjusting the one or more partitions to increase the size of an interface region that is supplied with oxygenation gas, the exposure of the blood to the oxygenation gas increases which, in turn, may increase the saturation of oxygen in the blood.

In some embodiments, the oxygenator comprises a gas-blood interface configured to be supplied with an oxygenation gas to expose the blood to the amount of oxygen. The gas-blood interface may, optionally, comprise a first interface region configured to receive a first oxygenation gas and a second interface region configured to receive a second oxygenation gas. The first interface region and the second interface region may collectively constitute the entirety of the gas-blood interface.

In other words, the gas-blood interface is divided into only two interface regions. It will therefore be appreciated that for whatever proportion of the gas-blood interface forms the first interface region, the remaining proportion of the gas-blood interface will form the second interface region. The first interface region may be smaller than, larger than, or equal in size to the second interface region.

For example, the gas-blood interface may be divided in half, such that each of the first interface region and the second interface region comprises 50% of the gas-blood interface (i.e. a ‘ 50/50 split’). In another example, the first interface region may comprise 40% of the gas-blood interface and the second interface region may comprise 60% of the gas-blood interface (i.e. a ‘ 40/60 split’), or vice versa (i.e. a ‘ 60/40 split’). Advantageously, the first interface region comprising 40% of the gas-blood interface provides appropriate regulation (e.g. addition, removal, and/or maintenance) of carbon dioxide when the first interface region is provided with an oxygenation gas at a standard flow rate (e.g. the oxygenation gas flow rate being approximately the same as the blood flow rate through the oxygenator, which may be around, for example, 4-5 litres per minute) that is commonly used during perfusion procedures. Other possible divides are also contemplated herein, such as: a 30/70 split, a 70/30 split, a 25/75 split, a 75/25 split, a 20/80 split, an 80/20 split, a 10/90 split, or a 90/10 split.

In some embodiments, the controller is configured to adjust the amount of oxygen to which the blood is exposed by, initially, solely supplying the first oxygenation gas to the first interface region without supplying the second oxygenation gas to the second interface region. The controller may further be configured to adjust the amount of oxygen by adjusting the amount of oxygen to which the blood is exposed by adjusting the concentration of oxygen in and/or the flow rate of the first oxygenation gas. The controller may further be configured to adjust the amount of oxygen by, subsequently, initiating supply of the second oxygenation gas to the second interface region to increase the amount of oxygen to which the blood is exposed.

The controller may be configured to increase and/or decrease the amount of oxygen to which the blood is exposed by increasing and/or decreasing the concentration of oxygen in and/or the flow rate of the first oxygenation gas.

Advantageously, this functionality allows the blood to be exposed initially to a lower amount of oxygen by supplying only a portion of the gas-blood interface (rather than supplying the entirety of the gas-blood interface). The blood is thus exposed to oxygenation gas only in the first interface region and is thus only able to exchange gases while in the first interface region of the gas-blood interface. This is particularly advantageous when treating a patient who is currently experiencing a reduced metabolic rate (e.g. a patient in circulatory arrest) and/or a reduced saturation of oxygen (e.g. a cyanotic patient). This functionality allows, for example, the patient's current saturation of oxygen to be chosen as the target saturation of oxygen (e.g. to initially match the treatment to the patient's current clinical state), even if the patient's saturation of oxygen is very low.

Initially, solely supplying the first oxygenation gas to the first interface region may comprise supplying a first oxygenation gas that consists of pure oxygen. Advantageously, supplying pure oxygen to only the first interface region (e.g. 40% of the gas-blood interface) provides a sufficiently high diffusion gradient for carbon dioxide between the first oxygenation gas and the blood passing through the oxygenator, thus ensuring adequate removal of carbon dioxide from the blood.

In particular, this functionality allows the system to expose the blood to a lower amount of oxygen (and thus operate at a lower saturation of oxygen) than if the entire gas blood interface were to be supplied with oxygenation gas. By way of example, supplying an entire gas-blood interface with oxygenation gas that contains 20% oxygen will expose the blood to more oxygen than if only 40% of the gas-blood interface were to be supplied with oxygenation gas that contains 20% oxygen.

Advantageously, this functionality further allows the system to gradually increase the saturation of oxygen by gradually increasing the concentration of oxygen in and/or the flow rate of the first oxygenation gas. That is, the controller may be configured to increase the amount of oxygen to which the blood is exposed by adjusting the concentration of oxygen in and/or the flow rate of the first oxygenation gas. This allows the amount of oxygen to which the blood is exposed to be gradually increased to thereby increase the saturation of oxygen in the patient's blood (i.e. as measured by the sensor).

At a certain point, the system can then begin supplying a second oxygenation gas to the second interface region to further increase the amount of oxygen to which the blood is exposed and thus further increase the saturation of oxygen in the patient's blood.

In general, the first oxygenation gas may be the same as, or different from, the second oxygenation gas. For example, the first oxygenation gas may be supplied from a first gas supply that is independent of a second gas supply that supplies the second oxygenation gas. This allows the flow rate and/or the composition of the first oxygenation gas and the second oxygenation gas to be adjusted independently. Alternatively, the first oxygenation gas and the second oxygenation gas may be supplied from a single gas supply. In this case, the controller may be configured to adjust the flow rate of the first oxygenation gas and the second oxygenation gas independently by controlling which interface regions are supplied by the single gas supply. However, in such an example, it may not be possible to alter the composition of the first oxygenation gas independently of the second oxygenation gas.

In some embodiments, the controller is configured to initiate supply of the second oxygenation gas responsive to the concentration of oxygen in the first oxygenation gas having been increased to a threshold concentration.

That is, the controller initially increases the concentration of oxygen in the first oxygenation gas until the concentration of oxygen reaches the threshold concentration, at which point the controller initiates the supply of the second oxygenation gas to the second interface region. For example, the threshold concentration may be 100%. In this case, the controller increases the concentration of oxygen in the first oxygenation gas until it can be increased no further. Thus, at this stage, the amount of oxygen to which the blood is exposed is at its maximum while only supplying gas to the first interface region. At this stage, the second oxygenation gas is supplied to the second interface region in order to continue increasing the amount of oxygen to which the blood is exposed. The threshold concentration need not be 100%, but could instead be some other value appropriate to a particular clinical situation.

In some embodiments, the controller is configured to adjust the amount of oxygen to which the blood is exposed by supplying the first oxygenation gas to the first interface region and supplying the second oxygenation gas to the second interface region. The controller may further be configured to adjust the amount of oxygen to which the blood is exposed by adjusting the concentration of oxygen in and/or the flow rate of the second oxygenation gas.

The controller may be configured to increase and/or decrease the amount of oxygen to which the blood is exposed by increasing and/or decreasing the concentration of oxygen in and/or the flow rate of the second oxygenation gas.

Advantageously, this functionality allows the saturation of oxygen to be adjusted while exposing the blood to a ‘high’ amount of oxygen in the oxygenator. That is, the entirety of the gas-blood interface is supplied with oxygenation gas. The blood is thus exposed to oxygenation gas in both the first interface region and the second interface region, and thus is able to exchange gases through the entirety of the gas-blood interface. This is particularly advantageous when a patient requites a ‘normal’ or ‘healthy’ saturation of oxygen (e.g. 98.5%).

In some embodiments, the controller is further configured to adjust the amount of oxygen to which the blood is exposed by reducing the concentration of oxygen in the first oxygenation gas responsive to the flow rate of the second oxygenation gas having been reduced to a threshold flow rate.

That is, the controller initially reduces the flow rate of the second oxygenation gas until the flow rate reaches the threshold flow rate, at which point the controller begins to reduce the concentration of oxygen of the first oxygenation gas. For example, the threshold flow rate may be zero (i.e. there is no longer a flow of the second oxygenation gas in the second interface region). In this case, the controller reduces the flow rate of the second oxygenation gas until it can be reduced no further. Thus, at this stage, the amount of oxygen to which the blood is exposed is at the minimum achievable level by controlling the flow rate of the second oxygenation gas alone. At this stage, the concentration of oxygen in the first oxygenation gas is reduced in order to continue reducing the amount of oxygen to which the blood is exposed. The threshold flow rate need not be zero, but could instead be some other value appropriate to the particular clinical situation.

In some embodiments, the target saturation of oxygen is a first target saturation of oxygen. The controller may be further configured to, responsive to the sensor measuring a saturation of oxygen equal to the first target saturation of oxygen, increase the concentration of oxygen in and/or the flow rate of the first oxygenation gas to reach a second target saturation of oxygen. Alternatively or additionally, the controller may be further configured to, responsive to the sensor measuring a saturation of oxygen equal to the first target saturation of oxygen, initiate supply of the second oxygenation gas to reach a second target saturation of oxygen. The second target saturation of oxygen may be higher than the first target saturation of oxygen.

The controller may be configured to increase and/or decrease the concentration of oxygen in and/or the flow rate of the first oxygenation gas to reach a first target saturation of oxygen.

The first target saturation of oxygen may be equal to a current saturation of oxygen of the patient's blood. The current saturation of oxygen of the patient's blood may be measured, for example by the sensor or by a peripheral sensor (e.g. a pulse oximeter) connected to the patient. Alternatively, the current saturation of oxygen of the patient's blood may be assumed (or estimated) based on known physiological parameters of the patient (e.g. an average saturation of oxygen based on known physiological parameters). In the case of a cyanotic patient, the first target saturation of oxygen may be 75% or below.

The second target saturation of oxygen may be equal to a desired saturation of oxygen of the patient's blood. For example, the desired saturation of oxygen may be chosen to be a saturation of oxygen that is typical of a healthy patient. For example, the second target saturation of oxygen may be in the range 95-100%, in the range 98-99%, or approximately 98.5%. Alternatively, the desired saturation of oxygen may be an intermediate value between the patient's current saturation of oxygen and the saturation of oxygen of a healthy patient, in order to more gradually increase the patient's saturation of oxygen. For example, the second target saturation of oxygen may be in the range 80-95%. The second target saturation of oxygen may be repeatedly adjusted (e.g. increased) until the patient's saturation of oxygen is that of a typical healthy patient.

The controller may be configured to receive the second target saturation of oxygen. More specifically, the second target saturation of oxygen may be received by the controller as an input. For example, the controller may be configured to receive the second target saturation of oxygen as an input from a user interface. Alternatively or additionally, the controller may be pre-programmed with a second target saturation of oxygen. Alternatively or additionally, the controller may be configured to retrieve the second target saturation of oxygen from a lookup table stored locally or on a remote server.

In some embodiments, the controller is configured to further adjust the amount of oxygen to which the blood is exposed by adjusting the concentration of oxygen in and/or the flow rate of the second oxygenation gas.

The controller may be configured to further increase and/or decrease the amount of oxygen to which the blood is exposed by increasing and/or decreasing (i) the concentration of oxygen in the second oxygenation gas, and/or (ii) the flow rate of the second oxygenation gas.

Advantageously, this allows the amount of oxygen to which the blood is exposed to be adjusted even after the first and second interface regions are both supplied with oxygenation gas. This, in turn, allows the concentration of oxygen to be increased up to 100% across both the first and second interface regions, thus increasing the amount of oxygen to which the blood is exposed up to its maximum possible value.

In some embodiments, the controller is further configured to, responsive to the sensor measuring a saturation of oxygen greater than the first target saturation of oxygen, decrease the flow rate of the second oxygenation gas. The controller may be further configured to, subsequently to decreasing the flow rate and responsive to the sensor measuring a saturation of oxygen that remains greater than the target saturation of oxygen, decrease the concentration of oxygen in the first oxygenation gas.

In some embodiments, the system further comprises a venous reservoir configured to receive blood from the patient. The system may optionally further comprise a pump configured to drive blood flow from the venous reservoir through the oxygenator.

The venous reservoir may be positioned upstream of the oxygenator. The venous reservoir may be configured to be positioned between the oxygenator and the patient. That is, the venous reservoir may receive deoxygenated blood from the patient. The pump may be configured to draw deoxygenated blood from the venous reservoir, and to cause the deoxygenated blood to flow into the oxygenator. The pump may be positioned upstream of the oxygenator. The pump may be positioned downstream of the venous reservoir. The pump may be positioned between the venous reservoir and the oxygenator. The pump may be a centrifugal pump or a roller (peristaltic) pump.

Also disclosed herein is a method of controlling blood oxygenation in a patient by an oxygenator that is configured to receive blood from the patient and to expose the blood to an amount of oxygen as the blood passes through the oxygenator to produce oxygenated blood.

The method comprises measuring, downstream of the oxygenator, a saturation of oxygen present in the oxygenated blood. The method further comprises calculating a difference between a target saturation of oxygen and the measured saturation of oxygen. The method further comprises adjusting the amount of oxygen to which the blood is exposed in the oxygenator to reduce the difference.

In some embodiments, the saturation of oxygen present in the oxygenated blood is measured on a blood line connected to the oxygenator and configured to carry the oxygenated blood from the oxygenator to the patient.

In some embodiments, measuring the saturation of oxygen comprises performing spectrophotometry on the blood the blood line.

In some embodiments, adjusting the amount of oxygen comprises adjusting the concentration of oxygen in an oxygenation gas received by the oxygenator.

In some embodiments, the oxygenation gas consists of pure oxygen.

In some embodiments, the oxygenator comprises a gas-blood interface which is supplied with an oxygenation gas to expose the blood to the amount of oxygen. Adjusting the amount of oxygen may comprise adjusting the proportion of the gas-blood interface that is supplied with the oxygenation gas.

In some embodiments, the gas-blood interface comprises a plurality of interface regions that are each configured to be independently supplied by a respective oxygenation gas. Adjusting the proportion of the gas-blood interface that is supplied with the oxygenation gas may comprise altering the number of interface regions that are supplied with the oxygenation gas.

In some embodiments, the oxygenator comprises a gas-blood interface configured to be supplied with an oxygenation gas to expose the blood to the amount of oxygen, and wherein the gas-blood interface comprises a first interface region configured to receive a first oxygenation gas and a second interface region configured to receive a second oxygenation gas, wherein the first interface region and the second interface region collectively constitute the entirety of the gas-blood interface.

In some embodiments, adjusting the amount of oxygen to which the blood is exposed comprises, initially, solely supplying the first oxygenation gas to the first interface region without supplying the second oxygenation gas to the second interface region. Adjusting the amount of oxygen to which the blood is exposed may further comprise adjusting the concentration of oxygen in and/or the flow rate of the first oxygenation gas. Adjusting the amount of oxygen may further comprise, subsequently, initiating supply of the second oxygenation gas to the second interface region of the gas-blood interface to increase the amount of oxygen to which the blood is exposed.

In some embodiments, initiating the supply of the second oxygenation gas is responsive to the concentration of oxygen in the first oxygenation gas having been increased to a threshold concentration.

In some embodiments, adjusting the amount of oxygen to which the blood is exposed comprises supplying the first oxygenation gas to the first interface region and supplying the second oxygenation gas to the second interface region. Adjusting the amount of oxygen to which the blood is exposed may further comprise adjusting the concentration of oxygen in and/or the flow rate of the second oxygenation gas.

In some embodiments, adjusting the amount of oxygen to which the blood is exposed comprises reducing the concentration of oxygen in the first oxygenation gas responsive to the flow rate of the second oxygenation gas having been reduced to a threshold flow rate.

In some embodiments, the target saturation of oxygen is a first target saturation of oxygen. The method may further comprise, responsive to measuring a saturation of oxygen equal to the first target saturation of oxygen, increasing the concentration of oxygen in and/or the flow rate of the first oxygenation gas to reach a second target saturation of oxygen. Alternatively or additionally, the method may further comprise, responsive to measuring a saturation of oxygen equal to the first target saturation of oxygen, initiating supply of the second oxygenation gas to reach a second target saturation of oxygen. The second target saturation of oxygen may be higher than the first target saturation of oxygen.

In some embodiments, the method further comprises further adjusting the amount of oxygen to which the blood is exposed by adjusting the concentration of oxygen in and/or the flow rate of the second oxygenation gas.

In some embodiments, the method further comprises, responsive to measuring a saturation of oxygen greater than the target saturation of oxygen, decreasing the flow rate of the second oxygenation gas. The method may further comprise, subsequently to decreasing the flow rate and responsive to measuring a saturation of oxygen that remains greater than the target saturation of oxygen, decreasing the concentration of oxygen in the first oxygenation gas.

Example embodiments will now be described with reference to the accompanying drawings in which:

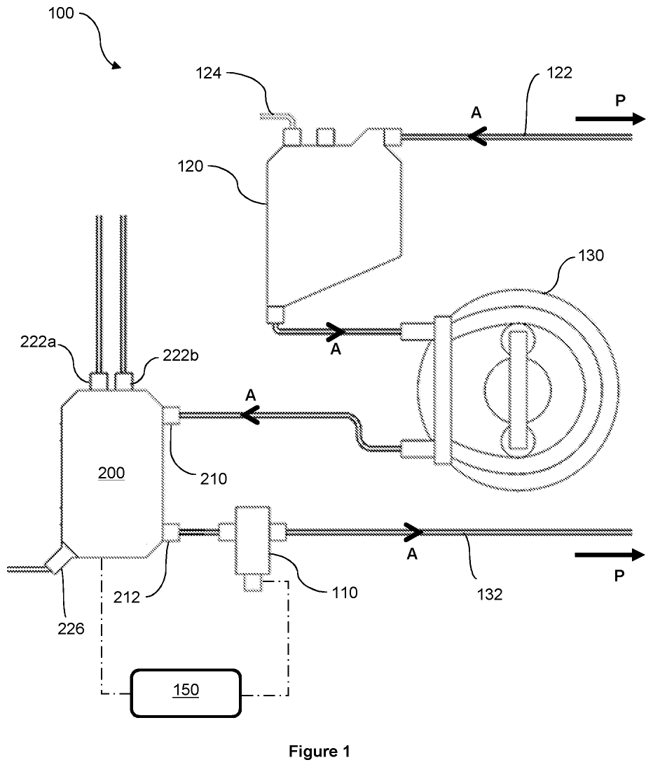

FIG. 1 is a schematic diagram of a system for controlling blood oxygenation in a patient;

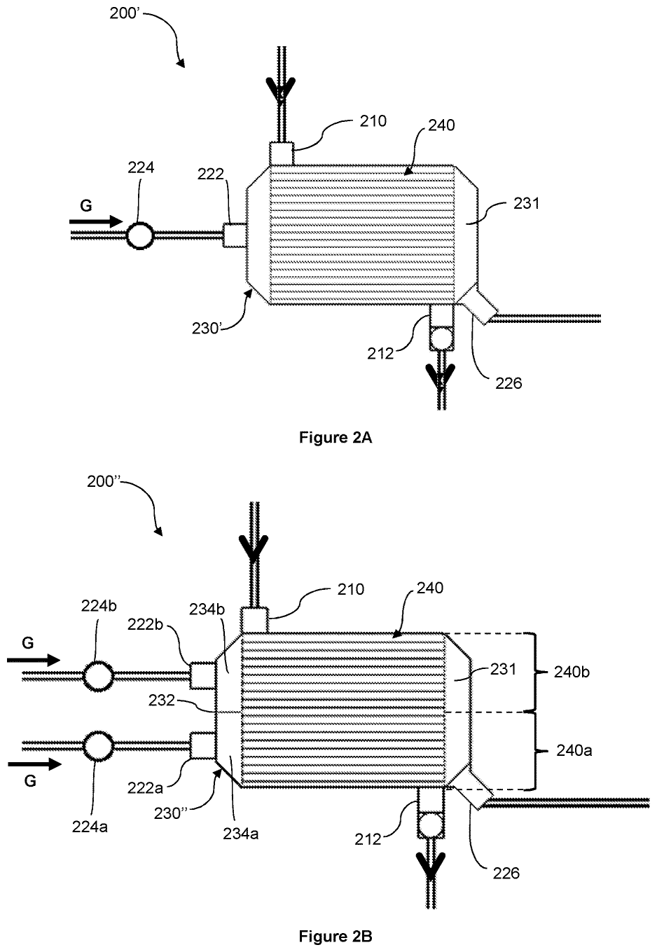

FIG. 2A is a schematic diagram of a first example of an oxygenator for a system for controlling blood oxygenation in a patient;

FIG. 2B is a schematic diagram of a second example of an oxygenator for a system for controlling blood oxygenation in a patient;

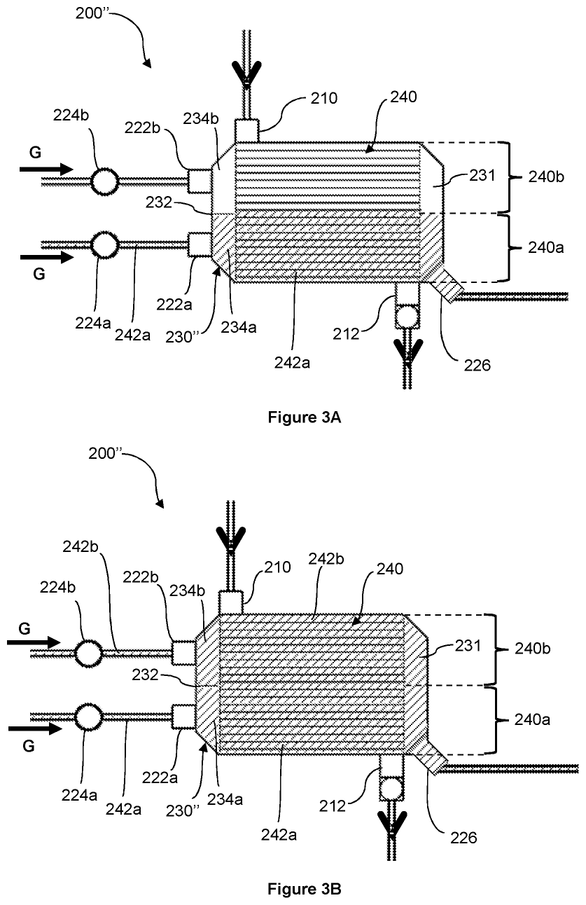

FIG. 3A is a schematic diagram of the oxygenator of FIG. 2B having a first interface region supplied with a first oxygenation gas;

FIG. 3B is a schematic diagram of the oxygenator of FIG. 2B having a first interface region supplied with a first oxygenation gas and a second interface region supplied with a second oxygenation gas;

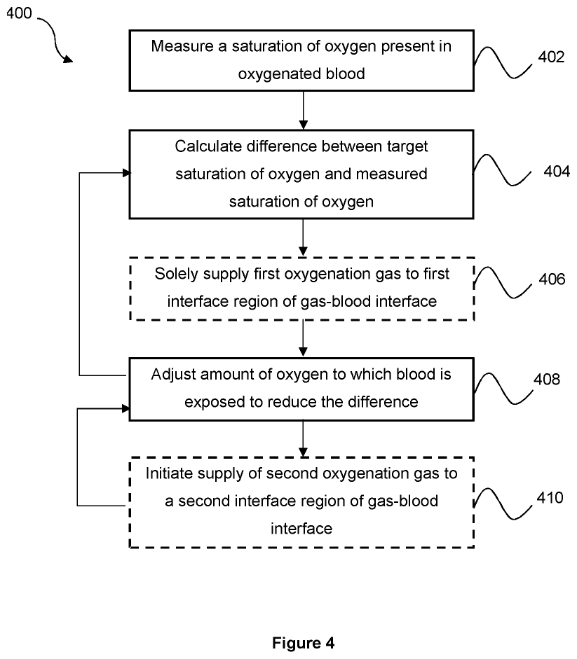

FIG. 4 is a flowchart of a first example of a method of controlling blood oxygenation in a patient by an oxygenator;

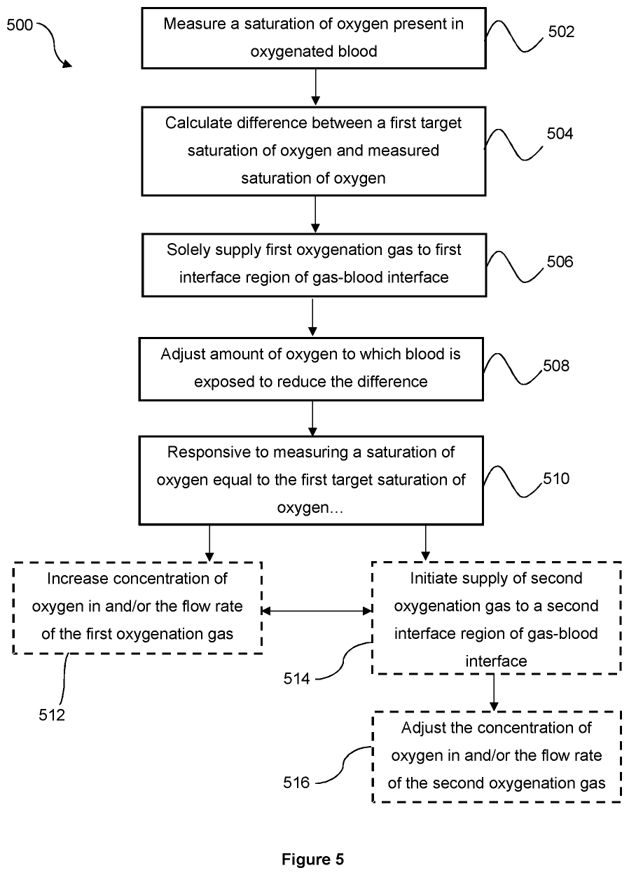

FIG. 5 is a flowchart of a second example of a method of controlling blood oxygenation in a patient by an oxygenator; and

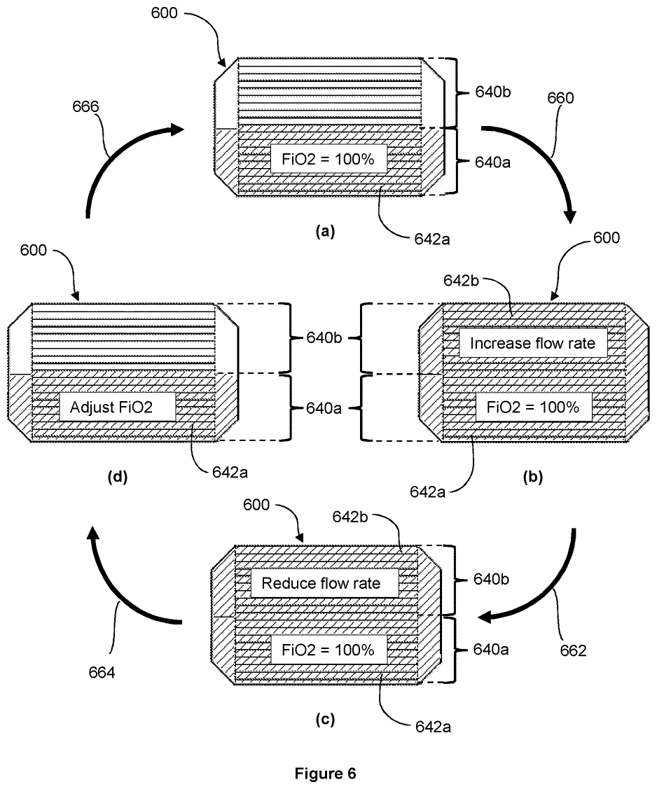

FIG. 6 is a schematic diagram illustrating states of operation of the oxygenator of FIG. 2B.

FIG. 1 depicts a system 100 for controlling blood oxygenation in a patient. The position of the patient relative to the system 100 is illustrated by arrows P, which indicate which blood lines (see below) lead to the patient. The system 100 comprises an oxygenator 200. The oxygenator 200 is configured to receive blood from the patient and to expose the blood to an amount of oxygen as the blood passes through the oxygenator 200 to produce oxygenated blood.

The system 100 further comprises a sensor 110. The sensor 110 is positioned downstream of the oxygenator 200. In the depicted example, the sensor 110 is positioned upstream of the patient on a blood line (in this case, arterial line 132) that leaves the oxygenator 200. The sensor 110 is configured to measure a saturation of oxygen present in the oxygenated blood (i.e. in the blood following oxygenation by the oxygenator 200). For example, the sensor 110 may be configured to measure the saturation of oxygen present in the oxygenated blood via spectrophotometry performed on the arterial line 132. In this case, the sensor 110 is considered to measure the ‘arterial saturation of oxygen’ (SaO2). It will be appreciated that other technologies for measuring the saturation of oxygen may be used in sensor 110.

The system 100 further comprises a controller 150. The controller 150 is configured to receive the measured saturation of oxygen from the sensor 110. The controller 150 is further configured to calculate a difference between a target saturation of oxygen and the measured saturation of oxygen. The controller 150 is further configured to adjust the amount of oxygen to which the blood is exposed in the oxygenator 200 to reduce the difference. The functionality of the controller 150 will be described in greater detail below, with reference to FIGS. 3A to 5.

The controller 150 is communicatively connected to the oxygenator 200 and the sensor 110, as depicted by the dash-dot lines in FIG. 1. That is, controller 150 may be configured to communicate with the oxygenator 200 and the sensor 110. This communication may occur via hardware connections (e.g. wired connections) or via wireless communication. In this regard, it will be understood that the dash-dot lines depicted in FIG. 1 are merely illustrative and do not necessarily represent physical connections between components. Furthermore, it will be understood that the controller 150 may be communicatively connected to more or fewer components in system 100. For example, controller 150 may not necessarily communicate directly with the oxygenator 200, but may instead be configured to communicate with valves, actuators and/or other gas flow control mechanisms that control the gas flow into and/or out of the oxygenator 200. Similarly, the controller 150 may not necessarily communicate directly with the sensor 110, but may instead be configured to communicate with an intermediate transceiver that relays measurements from the sensor 110 to the controller 150. The controller 150 may further be configured to communicate with the pump 130 and/or other components present in system 100. Other arrangements of communicative connections between components will be readily understood by the skilled person.

It is noted that ‘communication’ with the controller 150 refers to both the reception of data (e.g. measurements) by the controller 150 and the transmission of data (e.g. instructions or commands) by the controller 150.

The controller 150 may comprise any suitable type of data processing device, such as a microprocessor, a microcontroller or an application specific integrated circuit (ASIC). The data processing device may be communicatively coupled to a memory (e.g., a volatile memory, a non-volatile memory, or both volatile and non-volatile memories), upon which is stored processor-executable instructions that cause the controller to perform any of the methods disclosed herein.

As shown in FIG. 1, the system 100 is configured to receive blood from the patient via a venous line 122 and to return blood from the patient via an arterial line 132. The connection of the system 100 to the patient is illustrated by arrows P in FIG. 1. The system 100 further comprises a venous reservoir 120 that is configured to receive blood from the patient via the venous line 122. The venous reservoir 120 may additionally be configured to receive blood from the patient via one or more salvage lines, one or more purge lines, and/or one or more lines configured to carry surgical fluids (e.g. priming solutions, volume expanders, blood, and/or drugs), as represented by line 124 in FIG. 1. The venous reservoir 120 is positioned upstream of the oxygenator 200, between the patient and the oxygenator 200. It will be appreciated that, in other implementations, the exact arrangement of the venous reservoir 120 and the pump 130 may vary. In fact, the system 100 may not necessarily comprise the venous reservoir 120 and the pump 130. In such a case, the oxygenator 200 may be configured to receive the blood directly from the patient.

The blood, once collected in the venous reservoir 120, is driven through the oxygenator 200 by a pump 130. The pump 130 is located downstream of the venous reservoir 120 and upstream of the oxygenator 200. In the depicted embodiment, pump 130 is a roller (or peristaltic) pump. However, it will be appreciated that, depending on the circumstance, other types of pump may be used, such as a centrifugal pump. The blood exits the pump 130 and is received by the oxygenator 200 via a blood inlet 210. The blood is oxygenated by the oxygenator 200 and then exits the oxygenator via a blood outlet 212, before passing the sensor 110 and being returned to the patient via the arterial line 132. The blood flow path through the system 100 is shown by arrows A in FIG. 1.

The oxygenator 200 may take a variety of forms. Two examples of oxygenators that may be used in the present system are depicted in FIGS. 2A and 2B. Example structures of the oxygenator 200 will therefore now be described with reference to FIGS. 2A and 2B.

Starting with FIG. 2A, an oxygenator 200′ is depicted. As described above, the oxygenator 200′ comprises a blood inlet 210 for receiving blood from the patient and a blood outlet 212 for returning blood to the patient. The oxygenator 200′ further comprises a gas inlet 222 for receiving an oxygenation gas into the oxygenator 200′, as depicted by arrow G. The oxygenation gas enters the oxygenator 200′ from the gas inlet 222 via a gas inlet zone 230′. The oxygenator 200′ further comprises a gas outlet 226 (which may be referred to as a gas exhaust 226) for releasing waste gas from the oxygenator 200′. The oxygenation gas exits the oxygenator 200′ from the gas outlet 226 via a gas outlet zone 231.

The oxygenator 200′ further comprises a gas-blood interface 240. The gas inlet zone 230′ is fluidly connected with the gas-blood interface 240. The gas outlet zone 231 is also fluidly connected with the gas-blood interface 240. That is, the oxygenation gas enters the gas-blood interface 240 from the gas inlet zone 230′ and exits the gas-blood interface 240 via the gas outlet zone 231.

The gas-blood interface 240 may comprise one or more hollow fibre groups, each hollow fibre group comprising a plurality of hollow fibres. Each hollow fibre group comprises inlet potting in fluid connection with the gas inlet zone 230′. Each hollow fibre group comprises outlet potting in fluid connection with the gas outlet zone 231.

The gas-blood interface 240 is configured to be supplied with oxygenation gas to expose the blood to an amount of oxygen. For example, oxygenation gas may enter the hollow fibre groups via the inlet potting from the gas inlet zone 230′. The blood enters the oxygenator 200′ via the blood inlet 210. The blood and the oxygenation gas pass through the gas-blood interface 240 as they pass through the oxygenator 200′. The gas-blood interface 240 is configured to permit gaseous exchange between the blood and the oxygenation gas supplied to the gas-blood interface 240. This includes the transfer of gases (e.g. carbon dioxide, nitrogen) out of the blood and into the oxygenation gas as well as the transfer of gases (e.g. oxygen) into the blood and out of the oxygenation gas. Following gaseous exchange, the blood (now oxygenated) exits the oxygenator 200′, towards the patient, via blood outlet 212 and the oxygenation gas (now waste gas) exits the oxygenator 200′ via gas outlet 226.

The controller 150 may be configured to adjust the amount of oxygen to which the blood is exposed in the oxygenator by adjusting the concentration of oxygen in the oxygenation gas received by gas inlet 222 into the gas-blood interface 240. Alternatively or in addition, the controller 150 may be configured to adjust the amount of oxygen to which the blood is exposed in the oxygenator by adjusting the flow rate of the oxygenation gas through the oxygenator 200′. For example, as shown in FIG. 2A, the system 100 may further comprise a valve 224 that controls the flow of oxygenation gas into the gas inlet 222. The controller 150 may be configured to control the valve 224 (e.g. by opening or closing the valve 224) to adjust the amount of oxygenation gas supplied to the gas-blood interface 240.

Alternatively or in addition, the system may further comprise a gas blender (not shown) that is configured to blend one or more supply gases to produce one or more oxygenation gases for supplying to the oxygenator 200′. The controller 150 may be configured to adjust the composition (e.g. concentration of oxygen in) and/or the flow rate of the oxygenation gas by controlling the gas blender to do so.

Turning to FIG. 2B, oxygenator 200″ will now be described. Oxygenator 200″ is similar to oxygenator 200′, and like reference numerals are used for like features, where appropriate. For brevity, the description of these like features will not be repeated here. The difference between oxygenator 200′ and 200″ lies in the features that allow the supply of oxygenation gas to the oxygenator 200″.

Whereas oxygenator 200′ includes a single gas inlet 222 for receiving a single oxygenation gas into the oxygenator 200′, oxygenator 200″ comprises two gas inlets for receiving two oxygenation gases into the oxygenator 200″. Oxygenator 200″ comprises a first gas inlet 222a and a second gas inlet 222b. The first gas inlet 222a and the second gas inlet 222b are each fluidly connected to a gas inlet zone 230″. Gas inlet zone 230″ comprises a partition 232 that divides the gas inlet zone 230″ into a plurality of (in this case, two) gas inlet regions 234a, 234b. Each gas inlet region 234a, 234b is configured to receive a different one of the respective oxygenation gases. More specifically, a first gas inlet region 234a is configured to receive oxygenation gas from the first gas inlet 222a and the second gas inlet region 234b is configured to receive oxygenation gas from the second gas inlet 222b.

As can be seen in FIG. 2B, the presence of multiple gas inlet regions 234a, 234b effectively allows different proportions of the gas-blood interface 240 to be supplied with respective oxygenation gases. In this regard, the gas-blood interface 240 comprises a plurality of (in this case, two) interface regions 240a, 240b that are each configured to be independently supplied with a respective oxygenation gas. In such an example, the controller 150 may be configured to adjust the proportion of the gas-blood interface 140 that is supplied with oxygenation gas by altering the number of interface regions 240a, 240b that are supplied with oxygenation gas.

The partition 232 as shown in FIG. 2 divides the gas inlet zone 230″ into the first gas inlet region 234a and the second gas inlet region 234b that are of equal size. That is, the partition 232 divides the gas inlet zone 230″ (and thus the gas-blood interface 240) in half, such that each of the first gas inlet region 234a and the second gas inlet region 234b comprises 50% of the gas inlet zone 230″. In turn, the partition 232 thus acts to divide the gas-blood interface 240 in half such that each of the first interface region 240a and the second interface region 240b comprises 50% of the gas-blood interface 240. However, it will be appreciated that this divide is merely an example and the partition 232 (and/or multiple partitions) may be located at various positions in order to create different splits of the gas inlet zone 230″ and of the gas-blood interface 240. For example, the partition 232 may be positioned such that the first gas inlet region 234a comprises 40% of the gas inlet zone 230″ and the second gas inlet region 234b comprises 60% of the gas inlet zone 230″. That is, the gas-blood interface 240 may be divided such that the first interface region 240a comprises 40% of the gas-blood interface 240 and the second interface region 240b comprises 60% of the gas-blood interface 240.

The partition 232 as shown in FIG. 2 extends through the gas inlet zone 230″, but does not extend through the gas-blood interface 240. In such a case, the partition 232 may abut the inlet potting of the hollow fibre group to prevent gas flow between the interface regions 240a, 240b. Alternatively, the partition 232 may not abut the inlet potting because leakage of gas flow between the gas inlet regions 234a, 234b may be permissible. In other examples, the partition 232 may extend through the gas inlet zone 230″ and at least partially through the gas-blood interface 240 to physically separate the gas-blood interface 240 into interface regions 240a, 240b.

It will be noted that, in general, the oxygenation gases supplied to each of the gas inlet regions 234a, 234b (and thus to each of the interface regions 240a, 240b) may originate from the same or different gas supplies. The oxygenation gases may have the same composition or different compositions.

As described above, the system 100 may comprise one or more valves for controlling the flow of gas into the gas inlets of the oxygenator. In the case of oxygenator 200″, the system 100 may comprise a first valve 224a configured to control the flow of oxygenation gas into the first gas inlet 222a and a second valve 224b configured to control the flow of oxygenation gas into the second gas inlet 222b. The controller 150 may be configured to control (e.g. open or close) the first valve 224a and the second valve 224b independently of each other to adjust (e.g. increase or decrease) the flow rate through each of the interface regions 240a, 240b. Alternatively or additionally, the system 100 may comprise a gas blender for controlling the gas flow and/or composition, as described above. It will be appreciated that other examples of valves, actuators, blenders, gas flow controllers, or other components for controlling the flow rate and/or the composition of gases may be used instead of and/or in addition to the valves or gas blender described herein.

Turning to FIGS. 3A and 3B, the functionality of the oxygenator 200″ will be described. As already described, the partition 232 allows the gas inlet regions 234a, 234b to be independently supplied with respective oxygenation gases. Starting with FIG. 3A, the first gas inlet 222a may be supplied with a first oxygenation gas 242a. The first gas inlet 222a may be supplied with the first oxygenation gas 242a without supplying oxygenation gas to the second gas inlet 222b (i.e. oxygenation gas is solely supplied to the first gas inlet 222a). This supplies the first oxygenation gas 242a to the first gas inlet region 234a and thus to the first interface region 240a.

In this situation, the blood passing through the oxygenator 200″ is exposed to the first oxygenation gas 242a through only a portion (in this case, 50%) of the gas-blood interface 240. That is, while the blood passes through the second interface region 240b, the blood is exposed to substantially no oxygenation gas. The blood is only exposed to the first oxygenation gas 242a when the blood passes through the first interface region 240a. Therefore, the amount of oxygen to which the blood is exposed in the oxygenator 200″ can be kept low. The concentration and/or the flow rate of the first oxygenation gas 242a can be adjusted as described above to further adjust (e.g. increase and/or decrease) the amount of oxygen to which the blood is exposed. It will be appreciated that, depending on the relative size of the first interface region 240a and the second interface region 240b, a similar effect may be achieved by supplying oxygenation gas only to the second interface region 240b via the second gas inlet 222b.

Moving to FIG. 3B, the first oxygenation gas 242a is supplied to the first gas inlet 222a as in FIG. 3A and also a second oxygenation gas 242b is supplied to the second gas inlet 222b. This supplies the second oxygenation gas 242b to the second gas inlet region 234b and thus to the second interface region 240b.

In this situation, the blood passing through the oxygenator 200″ is exposed to the second oxygenation gas 242b when the blood passes through the second interface region 240b, and is subsequently exposed to the first oxygenation gas 242a when the blood passes through the first interface region 240a. Therefore, the amount of oxygen to which the blood is exposed in the oxygenator 200″ is increased relative to the situation depicted in FIG. 3A. The concentration and/or the flow rate of the first oxygenation gas 242a and/or the second oxygenation gas 242b can be adjusted as described above to further adjust (e.g. increase and/or decrease) the amount of oxygen to which the blood is exposed. It will be appreciated that the flow rate and/or the composition of the first oxygenation gas 242a can be adjusted independently of the flow rate and/or the composition of the second oxygenation gas 242b.

In this way, the controller 150 can use the oxygenator 200′ or the oxygenator 200″ to control the amount of oxygen to which the blood is exposed in the oxygenator.

The first oxygenation gas 242a and/or the second oxygenation gas 242b may consist of pure oxygen. The first oxygenation gas 242a and/or the second oxygenation gas 242b may comprise a mixture of oxygen and nitrogen.

While specific examples of oxygenators that may be used with the system 100 have been described, it will be appreciated that other oxygenators may be used that achieve the same or similar functionality. For example, an oxygenator may comprise multiple gas inlet zones that each connect to a separate gas inlet. Alternatively, the gas inlet(s) may connect directly to the gas-blood interface, which may be separated into a plurality of interface regions within the oxygenator. It will be appreciated that other structures of oxygenator exist that allow for a plurality of interface regions to be independently supplied with a respective oxygenation gas. Furthermore, while oxygenators have been shown with one or two gas inlet regions and one or two interface regions, it will be appreciated that substantially any number of gas inlet regions and interface regions may be present. The more gas inlet regions and interface regions that are present, the more independent oxygenation gases that can be supplied to the gas-blood interface.

The functionality of the system 100 as a whole will now be described with reference to FIGS. 4 and 5.

FIG. 4 depicts a first example method 400 of controlling blood oxygenation in a patient, as contemplated herein. The controller 150 may be configured to perform any or all of the operations of the method 400. The method 400 comprises measuring 402 a saturation of oxygen present in the oxygenated blood. The measuring 402 may, for example, be performed by the sensor 110. The method 400 further comprises calculating 404 a difference between a target saturation of oxygen and the measured saturation of oxygen. The method 400 further comprises adjusting 408 the amount of oxygen to which the blood is exposed to reduce the difference. The adjusting 408 may comprise adjusting the composition of (e.g. the concentration of oxygen in) and/or the flow rate of the oxygenation gas (or gases) supplied to the oxygenator, as described above with reference to FIGS. 2A-3B. Following the adjusting 408 of the amount of oxygen to which the blood is exposed, the method 400 may comprise recalculating the difference between the target saturation of oxygen and the measured saturation of oxygen. The adjusting 408 and the calculating 404 may therefore be repeated and/or performed continuously, as indicated by the return arrow joining these boxes in FIG. 4.

Optionally, the method 400 (e.g. the adjusting 408) may further comprise solely supplying 406 the first oxygenation gas 242a to the first interface region 240a of the gas-blood interface 240. The adjusting 408 may comprise adjusting the composition of (e.g. the concentration of oxygen in) and/or the flow rate of the first oxygenation gas 242a.

Optionally, the method 400 (e.g. the adjusting 408) may further comprise initiating 410 supply of the second oxygenation gas 242b to the second interface region 240b of the gas-blood interface 240. The adjusting 408 may comprise adjusting the composition of (e.g. the concentration of oxygen in) and/or the flow rate of the second oxygenation gas 242b.

For example, initiating 410 the supply of the second oxygenation gas 242b may be responsive to the concentration of oxygen in the first oxygenation gas 242a having been increased to a threshold concentration (e.g. 100% oxygen).

In an example scenario, oxygenation gas (in the form of the first oxygenation gas 242a) may initially be supplied solely to the first interface region 240a. At this stage, the first oxygenation gas 242a may comprise a mixture of nitrogen and oxygen. In this regard, the blood is exposed to a ‘low’ amount of oxygen (and thus a ‘low’ saturation of oxygen in the patient's blood can be achieved). The concentration of oxygen in the first oxygenation gas 242a may be gradually increased to increase the amount of oxygen to which the blood is exposed (and thus increase the saturation of oxygen in the blood). Once the concentration of oxygen in the first oxygenation gas 242a reaches 100%, it is no longer possible to increase the amount of oxygen to which the blood is exposed by increasing the concentration of oxygen in the first oxygenation gas 242a. Therefore, supply of the second oxygenation gas 242b is initiated to further increase the amount of oxygen to which the blood is exposed. The concentration of oxygen in and/or the flow rate of the second oxygenation gas 242b can then be further adjusted to adjust the amount of oxygen to which the blood is exposed.

While, for ease of illustration, operations 406, 408, and 410 in FIG. 4 have been depicted as sequential, it will be appreciated that these operations are interlinked and, for example, it may be considered that the adjusting 408 comprises the supplying 406 and the initiating 410.

FIG. 5 depicts a second example method 500 of controlling blood oxygenation in a patient, as contemplated herein. The controller 150 may be configured to perform any or all of the operations of the method 500. The method 500 comprises measuring 502 a saturation of oxygen present in the oxygenated blood. The measuring 502 may, for example, be performed by the sensor 110. The method 500 further comprises calculating 504 a difference between a first target saturation of oxygen and the measured saturation of oxygen. The method 500 further comprises solely suppling 506 the first oxygenation gas 242a to the first interface region 240a of the gas-blood interface 240. The method 500 further comprises adjusting 508 the amount of oxygen to which the blood is exposed to reduce the difference. The adjusting 508 may comprise adjusting the composition of (e.g. the concentration of oxygen in) and/or the flow rate of the oxygenation gas (or gases) supplied to the oxygenator, as described above with reference to FIGS. 2A-3B. For example, the adjusting 508 may comprise adjusting the composition of (e.g. the concentration of oxygen in) and/or the flow rate of the first oxygenation gas 242a.

The method 500 further comprises, responsive to measuring 510 (e.g. by sensor 110) a saturation of oxygen equal to the first target saturation of oxygen: increasing 512 the concentration of oxygen in and/or the flow rate of the first oxygenation gas 242a; and/or initiating 514 supply of the second oxygenation gas 242b to the second interface region 240b of the gas-blood interface 240. The increasing 512 and/or the initiating 514 may be performed to reach a second target saturation of oxygen. The second target saturation of oxygen may be higher than the first target saturation of oxygen. The method 500 may further comprise adjusting 516 the concentration of oxygen in and/or the flow rate of the second oxygenation gas 242b to reach the second target saturation of oxygen.

In an example scenario, oxygenation gas (in the form of the first oxygenation gas 242a) is initially solely supplied to the first interface region 240a. At this stage, the first oxygenation gas 242a may comprise a mixture of nitrogen and oxygen. In this regard, a low amount of oxygen (and thus a low saturation of oxygen in the patient's blood can be achieved). The concentration of oxygen in the first oxygenation gas 242a may be gradually increased to increase the amount of oxygen to which the blood is exposed (and thus increase the saturation of oxygen in the blood) until the saturation of oxygen (as measured by sensor 110) reaches the first target saturation of oxygen (i.e. there is no difference between the first target saturation of oxygen and the measured saturation of oxygen). At this stage, a second (e.g. higher) target saturation of oxygen may be received or retrieved by the controller 150 (or be chosen by a clinician). To achieve the second target saturation of oxygen, the concentration of oxygen in and/or the flow rate of the first oxygenation gas 242a in the first interface region 240a may be increased and/or the supply of the second oxygenation gas 242b to the second interface region 240b may be initiated. The concentration of oxygen in and/or the flow rate of the second oxygenation gas 242b can then be further adjusted to adjust the amount of oxygen to which the blood is exposed.

The method 500 may be particularly useful in the treatment of cyanotic patients. Cyanotic patients are those suffering from low saturation of oxygen in their blood. During surgery and/or treatment of a cyanotic patient, it is desirable to increase the saturation of oxygen in the patient's blood. However, oxygenating the patient's blood to a high oxygen saturation too quickly can cause hyperoxia, which can lead to physiological damage to the patient. Therefore, in method 500, the first target saturation of oxygen may be chosen to be equal to, similar to, or marginally greater than (e.g. by 1-2%) the patient's current saturation of oxygen, which may be around 75% in a cyanotic patient. The saturation of oxygen can be maintained and gradually increased using the closed-loop functionality as described herein. A second target saturation of oxygen can then be chosen that is higher (e.g. marginally higher, such as by 1-2%) than the current saturation of oxygen. The system 100 will then increase the saturation in the blood (i.e. adjust the amount of oxygen to which the blood is exposed to reduce the difference). The second target saturation of oxygen can be repeatedly and incrementally increased until the target saturation of oxygen is a saturation of oxygen typical of a healthy patient (e.g. 98-100%). Thereby, the patient is brought from a state of cyanosis up to a healthy saturation of oxygen.

The concentration of oxygen in the first oxygenation gas 242a and in the second oxygenation gas 242b may be increased to substantially 100% (i.e. pure oxygen). This has the advantage of maximising the removal of nitrogen from the patient's blood which, in turn, reduces the risk of harmful GME forming in the patient's blood. In particular, this increase to 100% may be performed as a final operation during the treatment of the patient to ensure the removal of GME before the treatment is concluded.

Turning to FIG. 6, an example of the operation of the system as described herein will be described. FIG. 6 shows a schematic representation of several ‘states’ that may exist in the operation of the system described herein. In particular, FIG. 6 illustrates a series of states that may occur during the treatment of a patient who experiences a low metabolic rate (e.g. during circulatory arrest).

FIG. 6 is divided into a series of four states, (a)-(d), which will be described in sequence below. The transitions between the states are illustrated by arrows 660, 662, 664, and 666. In each state, an oxygenator 600 is depicted. For clarity, the details of oxygenator 600 will not be described and are not labelled in FIG. 6. However, it will be appreciated that oxygenator 600 may share any or all of the features of oxygenator 200″, as described above. The only features labelled in FIG. 6 are the first interface region 640a, the second interface region 640b, the first oxygenation gas 642a, and the second oxygenation gas 642b. The following description will begin with state (a), but it will be appreciated that the process is cyclic and so any state may be considered the ‘start’.

In state (a), the first interface region 640a is supplied with the first oxygenation gas 642a. The second interface region 640b is not supplied with oxygenation gas. As depicted in FIG. 6, the first oxygenation gas 642a is supplied with an FiO2 of 100%. That is, in state (a), the first oxygenation gas 642a consists solely of oxygen. The oxygenator 600 may be operated in this state in order to remove carbon dioxide from the blood of the patient, while also providing oxygen to the blood. In this regard, the first oxygenation gas 642a may be considered a ‘sweep gas’. However, given that the first interface region 640a represents only a proportion (e.g. 40%) of the overall gas-blood interface, the supply of the first oxygenation gas 642a may not be sufficient to reach a target saturation of oxygen in the patient's blood. For example, the sensor may measure a saturation of oxygen below the target saturation of oxygen. The system may then transition to state (b), as indicated by arrow 660.

In state (b), the first interface region 640a continues to be supplied with the first oxygenation gas 642a, at 100% FiO2. However, the second interface region 640b now begins to be supplied with the second oxygenation gas 642b. That is, the flow rate of the second oxygenation gas 642b may be increased from zero. The flow rate of the second oxygenation gas 642b may continue to be increased until the target saturation of oxygen in the patient's blood is reached (e.g. until the sensor measures a saturation of oxygen equal to the target saturation of oxygen). It will be appreciated that supplying the second interface region 640b with the second oxygenation gas 242b increases the amount of oxygen to which the blood is exposed beyond what is possible by only supplying the first interface region 640a. Therefore, a higher saturation of oxygen may be achieved by supplying both interface regions 640a, 640b. The second oxygenation gas 642b may be provided at 100% FiO2 (i.e. pure oxygen). The concentration of oxygen in the second oxygenation gas 642b may be adjusted in addition to or instead of adjusting the flow rate of the second oxygenation gas 642b. The system may operate in this state to increase the saturation of oxygen in the patient's blood to a healthy level (e.g. 98.5%). However, during a surgical procedure, the patient's metabolic rate may drop. This, in turn, means that the blood needs to be exposed to a lower amount of oxygen in the oxygenator to achieve the same saturation of oxygen in the patient's blood. This drop in metabolic rate may be detected in the form of an increase in the saturation of oxygen in the patient's blood caused by the continual supply of the first and second oxygenation gases 642a, 642b at the same oxygen concentration and flow rate in spite of the drop in metabolic rate. This may be detected, for example, by the sensor. In such a case, the system may then transition to state (c), as indicated by arrow 662.

In state (c), the flow rate of the second oxygenation gas 642b is reduced. This reduces the volume of the second oxygenation gas 642b present in the second interface region 640b at a given time. The amount of oxygen to which the blood is exposed is thus reduced, leading to a decrease in the saturation of oxygen in the patient's blood. The saturation of oxygen may return to the target saturation of oxygen (e.g. 98.5%) at a lower flow rate of the second oxygenation gas 642b. Alternatively, the flow rate of the second oxygenation gas 642b may need to be reduced to zero (such that there is no oxygenation gas in the second interface region 640b) before the target saturation of oxygen can be achieved. In some instances, it may be that this reduction to zero still is not a sufficient reduction to achieve the target saturation of oxygen. Therefore, to continue to reduce the amount of oxygen to which the blood is exposed (e.g. in response to the flow rate of the second oxygenation gas 642b being reduced to zero), the system may then transition to state (d), as indicated by arrow 664.

In state (d), there is no supply of oxygenation gas to the second interface region 640b. To further reduce the amount of oxygen to which the blood is exposed, the concentration of oxygen in the first oxygenation gas 642a may be reduced. This further reduces the amount of oxygen in the oxygenator 600 and thus allows the saturation of oxygen to be further reduced until the saturation of oxygen reaches the target saturation of oxygen. Advantageously, this allows the system to effectively manage the saturation of oxygen in a patient who is experiencing a low metabolic rate (e.g. a patient in circulatory arrest). As the patient returns to a higher (e.g. normal) metabolic rate, the FiO2 of the first oxygenation gas 642a can be increased again to increase the amount of oxygen present in the oxygenator 600. The FiO2 can continue to be increased until it reaches 100%. The system thereby transitions back to state (a), as indicated by arrow 666.

It will be appreciated that FIG. 6 represents a particular example of the operation of the systems described herein in a patient whom initially has a normal metabolic rate and subsequently has a low metabolic rate. In practice, a patient's metabolic rate may increase or decrease during a surgical procedure and thus the system may move freely between all of the states in FIG. 6. That is, the system is not restricted merely to the order described but instead may increase and/or decrease the concentration and/or flow rate of the first and/or second oxygenation gases as needed to achieve the target saturation of oxygen (or other physiological parameter).

It will be understood that any features, functions, characteristics, or advantages described with respect to the above-mentioned examples of a system may be applied to the above-mentioned examples of methods, and vice versa. Similarly, the features, functions, characteristics, or advantages described with respect to the above-mentioned examples of oxygenators may be applied to any of the above-mentioned examples of systems or methods.

In accordance with the present disclosure, the following aspects are also contemplated.

Aspect 1. A method of controlling blood oxygenation in a patient by an oxygenator that is configured to receive blood from the patient and to expose the blood to an amount of oxygen as the blood passes through the oxygenator to produce oxygenated blood, the method comprising:
measuring, downstream of the oxygenator, a saturation of oxygen present in the oxygenated blood;
calculating a difference between a target saturation of oxygen and the measured saturation of oxygen; and
adjusting the amount of oxygen to which the blood is exposed in the oxygenator to reduce the difference.

Aspect 2. The method of Aspect 1, wherein the saturation of oxygen present in the oxygenated blood is measured on a blood line connected to the oxygenator and configured to carry the oxygenated blood from the oxygenator to the patient.

Aspect 3. The method of Aspect 2, wherein measuring the saturation of oxygen comprises performing spectrophotometry on the oxygenated blood in the blood line.

Aspect 4 The method of any of the preceding Aspects, wherein adjusting the amount of oxygen comprises adjusting the concentration of oxygen in an oxygenation gas received by the oxygenator.

Aspect 5. The method of any of the preceding Aspects, wherein the oxygenation gas consists of pure oxygen.

Aspect 6. The method of any of the preceding Aspects, wherein the oxygenator comprises a gas-blood interface which is supplied with an oxygenation gas to expose the blood to the amount of oxygen, and wherein adjusting the amount of oxygen comprises adjusting the proportion of the gas-blood interface that is supplied with the oxygenation gas.

Aspect 7. The method of Aspect 6, wherein the gas-blood interface comprises a plurality of interface regions that are each configured to be independently supplied by a respective oxygenation gas, and wherein adjusting the proportion of the gas-blood interface that is supplied with oxygenation gas comprises altering the number of interface regions that are supplied with oxygenation gas.

Aspect 8. The method of any of the preceding Aspects, wherein the oxygenator comprises a gas-blood interface configured to be supplied with an oxygenation gas to expose the blood to the amount of oxygen, and wherein the gas-blood interface comprises a first interface region configured to receive a first oxygenation gas and a second interface region configured to receive a second oxygenation gas, wherein the first interface region and the second interface region collectively constitute the entirety of the gas-blood interface.

Aspect 9. The method of Aspect 8, wherein adjusting the amount of oxygen to which the blood is exposed comprises:
initially, solely supplying the first oxygenation gas to the first interface region without supplying the second oxygenation gas to the second interface region;
adjusting the amount of oxygen to which the blood is exposed by adjusting the concentration of oxygen in and/or the flow rate of the first oxygenation gas; and
subsequently, initiating supply of the second oxygenation gas to the second interface region of the gas-blood interface to increase the amount of oxygen to which the blood is exposed.

Aspect 10. The method of claim Aspect 9, wherein initiating the supply of the second oxygenation gas is responsive to the concentration of oxygen in the first oxygenation gas having been increased to a threshold concentration.

Aspect 11. The method of any of Aspects 8 to 10, wherein adjusting the amount of oxygen to which the blood is exposed comprises:
supplying the first oxygenation gas to the first interface region and supplying the second oxygenation gas to the second interface region; and
adjusting the amount of oxygen to which the blood is exposed by adjusting the concentration of oxygen in and/or the flow rate of the second oxygenation gas.

Aspect 12. The method of Aspect 11, wherein adjusting the amount of oxygen to which the blood is exposed comprises reducing the concentration of oxygen in the first oxygenation gas responsive to the flow rate of the second oxygenation gas having been reduced to a threshold flow rate.

Aspect 13. The method of any of Aspects 8 to 12, wherein the target saturation of oxygen is a first target saturation of oxygen, and wherein the method further comprises:
responsive to measuring a saturation of oxygen equal to the first target saturation of oxygen,
   i) increasing the concentration of oxygen in and/or the flow rate of the first oxygenation gas; and/or
   ii) initiating supply of the second oxygenation gas, to reach a second target saturation of oxygen, the second target saturation of oxygen being higher than the first target saturation of oxygen.

Aspect 14. The method of any of Aspects 8 to 13, further comprising further adjusting the amount of oxygen to which the blood is exposed by adjusting the concentration of oxygen in and/or the flow rate of the second oxygenation gas.

Aspect 15. The method of any of aspects 8 to 14, wherein the method further comprises:
responsive to measuring a saturation of oxygen greater than the target saturation of oxygen, decreasing the flow rate of the second oxygenation gas; and
subsequently to decreasing the flow rate and responsive to the sensor measuring a saturation of oxygen that remains greater than the target saturation of oxygen, decreasing the concentration of oxygen in the first oxygenation gas.

1. A system for controlling blood oxygenation in a patient, the system comprising:
an oxygenator configured to receive blood from the patient and to expose the blood to an amount of oxygen as the blood passes through the oxygenator to produce oxygenated blood;
a sensor positioned downstream of the oxygenator and configured to measure a saturation of oxygen present in the oxygenated blood; and
a controller configured to
receive the measured saturation of oxygen from the sensor,
calculate a difference between a target saturation of oxygen and the measured saturation of oxygen, and
adjust the amount of oxygen to which the blood is exposed in the oxygenator to reduce the difference.

2. The system of claim 1, wherein the sensor is positioned on a blood line, the blood line being connected to the oxygenator and configured to carry the oxygenated blood from the oxygenator to the patient.

3. The system of claim 2, wherein the sensor is configured to measure the saturation of oxygen present in the oxygenated blood via spectrophotometry performed on the blood line.

4. The system of claim 1, wherein the controller is configured to adjust the amount of oxygen by adjusting the concentration of oxygen in an oxygenation gas received by the oxygenator.

5. The system of claim 1, wherein the controller is configured to adjust the amount of oxygen by adjusting the flow rate of an oxygenation gas received by the oxygenator.

6. The system of claim 1, wherein the oxygenation gas consists of pure oxygen.

7. The system of claim 1, wherein the oxygenator comprises a gas-blood interface configured to be supplied with an oxygenation gas to expose the blood to the amount of oxygen, and wherein the controller is configured to adjust the amount of oxygen by adjusting the proportion of the gas-blood interface that is supplied with the oxygenation gas.

8. The system of claim 7, wherein the gas-blood interface comprises a plurality of interface regions that are each configured to be independently supplied with a respective oxygenation gas, and wherein the controller is configured to adjust the proportion of the gas-blood interface that is supplied with oxygenation gas by altering the number of interface regions that are supplied with oxygenation gas.

9. The system of claim 8, wherein the oxygenator comprises a gas inlet zone for receiving oxygenation gas into the gas-blood interface, wherein the gas inlet zone comprises one or more partitions dividing the gas inlet zone into a plurality of gas inlet regions, each gas inlet region being configured to receive oxygenation gas from a different one of the respective oxygenation gases and to provide the respective oxygenation gas to the respective interface region.

10. The system of claim 1, wherein the oxygenator comprises a gas-blood interface configured to be supplied with an oxygenation gas to expose the blood to the amount of oxygen, and wherein the gas-blood interface comprises a first interface region configured to receive a first oxygenation gas and a second interface region configured to receive a second oxygenation gas, wherein the first interface region and the second interface region collectively constitute the entirety of the gas-blood interface.

11. The system of claim 10, wherein the controller is configured to adjust the amount of oxygen to which the blood is exposed by:
initially, solely supplying the first oxygenation gas to the first interface region without supplying the second oxygenation gas to the second interface region;
adjusting the amount of oxygen to which the blood is exposed by adjusting the concentration of oxygen in and/or the flow rate of the first oxygenation gas; and
subsequently, initiating supply of the second oxygenation gas to the second interface region to increase the amount of oxygen to which the blood is exposed.

12. The system of claim 11, wherein the controller is configured to initiate supply of the second oxygenation gas responsive to the concentration of oxygen in the first oxygenation gas having been increased to a threshold concentration.

13. The system of claim 10, wherein the controller is configured to adjust the amount of oxygen to which the blood is exposed by:
supplying the first oxygenation gas to the first interface region and supplying the second oxygenation gas to the second interface region; and
adjusting the amount of oxygen to which the blood is exposed by adjusting the concentration of oxygen in and/or the flow rate of the second oxygenation gas.

14. The system of claim 13, wherein the controller is further configured to adjust the amount of oxygen to which the blood is exposed by reducing the concentration of oxygen in the first oxygenation gas responsive to the flow rate of the second oxygenation gas having been reduced to a threshold flow rate.

15. The system of claim 10, wherein the target saturation of oxygen is a first target saturation of oxygen, and wherein the controller is further configured to:
responsive to the sensor measuring a saturation of oxygen equal to the first target saturation of oxygen,
i) increase the concentration of oxygen in and/or the flow rate of the first oxygenation gas; and/or
ii) initiate supply of the second oxygenation gas,
to reach a second target saturation of oxygen, the second target saturation of oxygen being higher than the first target saturation of oxygen.

16. The system of claim 10, wherein the controller is configured to further adjust the amount of oxygen to which the blood is exposed by adjusting the concentration of oxygen in and/or the flow rate of the second oxygenation gas.

17. The system of claim 10, wherein the controller is further configured to:
responsive to the sensor measuring a saturation of oxygen greater than the target saturation of oxygen, decrease the flow rate of the second oxygenation gas; and
subsequently to decreasing the flow rate and responsive to the sensor measuring a saturation of oxygen that remains greater than the target saturation of oxygen, decrease the concentration of oxygen in the first oxygenation gas.

18. The system of claim 10, further comprising:
a venous reservoir configured to receive blood from the patient; and
a pump configured to drive blood flow from the venous reservoir through the oxygenator.
